(19) United States
(12) Patent Application Publication
(10) Pub. No.: US 2022/0280916 A1
(43) Pub. Date: Sep. 8, 2022

(54) Liquid-Absorbing Material And Preparation Method Thereof, Absorbent Core And Absorbent Product

(71) Applicant: 3M INNOVATIVE PROPERTIES COMPANY, St. Paul, MN (US)

(72) Inventors: Miaoru Ling, Guangzhou (CN); You San Qian, Shanghai (CN); Rui Chen, Shanghai (CN); Jun Cheng, Guangzhou (CN)

(21) Appl. No.: 17/753,184

(22) PCT Filed: Aug. 25, 2020
(86) PCT No.: PCT/IB2020/057937

§ 371 Date: Feb. 23, 2022

(30) Foreign Application Priority Data
Aug. 30, 2019 (CN) 201910813506.7

(51) Int. Cl. B01J 20/26; B01J 20/30; B01J 20/28

(52) U.S. Cl. CPC B01J 20/261; B01J 20/3078; B01J 20/28016

(57) ABSTRACT

The present invention provides a liquid-absorbing material and a preparation method thereof, an absorbent core and an absorbent product, and belongs to the technical field of liquid-absorbing materials. The present invention can at least partially address the problem of poor absorption performance of existing absorbent cores. The liquid-absorbing material of the present invention comprises: polymer fiber, comprising a polymer fiber comprising a polymer material capable of melting when heated, wherein the polymer fiber has been partially molted; and an absorbent particle having liquid absorbing capacity, wherein when the polymer fiber is in the partially molten state, the absorbent particle is in contact with the polymer fiber, thereby bonding to the polymer fiber after the polymer fiber is cured.

### TECHNICAL FIELD

The present invention belongs to the technical field of liquid-absorbing materials, and particularly relates to a liquid-absorbing material and a preparation method thereof, an absorbent core and an absorbent product.

### BACKGROUND

In products such as disposable diapers and sanitary napkins, an absorbent core constitutes the core structure. Conventional absorbent cores comprise pulp fibers of, for example, wood and cotton, but these fibers have poor absorption performance. For this reason, super absorbent polymer (SAP) absorbent particles can be added to the pulp fibers of an absorbent core, but the amount of addition is limited, causing problems such as gel blocking and separation of absorbent particles.

Therefore, the absorption performance of existing absorbent cores is poor.

### SUMMARY

The present invention at least partially addresses the problem of poor absorption performance of existing absorbent cores.

One aspect of the present invention provides a liquid-absorbing material, comprising: a polymer fiber, comprising a polymer material capable of melting when heated, wherein the polymer fiber has been partially molted; and

an absorbent particle having liquid absorbing capability, wherein when the polymer fiber is in a partially molten state, the absorbent particle is in contact with the polymer fiber, thereby bonding to the polymer fiber after the polymer fiber is cured.

Optionally, the polymer fiber comprises a fiber web, and the fiber web comprises at least one of a spunlaid fiber web, a hot air-laid fiber web and a meltblown fiber web.

Optionally, the polymer fiber has a fineness between 1 micrometer and 30 micrometers.

Optionally, the polymer fiber comprises polypropylene elastomeric fibers or polyester elastomeric fibers.

Optionally, the absorbent particle comprises a super absorbent polymer particle.

Optionally, the super absorbent polymer particle comprises sodium polyacrylate.

Optionally, the absorbent particle has a particle size between 200 micrometers and 600 micrometers.

Optionally, the liquid-absorbing material comprises 1 wt % to 95 wt % of the absorbent particle.

Optionally, the liquid-absorbing material comprises 70 wt % to 90 wt % of the absorbent particle.

Optionally, the absorbent particle is composed of a high polymer material, and the absorbent particle has a melting point higher than that of the polymer fiber.

One aspect of the present invention provides an absorbent core, comprising:

an absorbent layer consisting of any of the aforementioned liquid-absorbing materials.

Optionally, the absorbent layer has a thickness between 0.2 mm and 30 mm.

Optionally, the absorbent layer has a grammage between 10 gsm and 1000 gsm.

Optionally, the absorbent core further comprises:

a hydrophilic nonwoven fabric and a hydrophobic nonwoven fabric respectively arranged on two sides of the absorbent layer.

One aspect of the present invention provides an absorbent product, comprising:

any of the aforementioned absorbent cores.

Optionally, the absorbent product is any one of a disposable nappy, a disposable diaper, a pair of disposable training pants, a sanitary napkin and a napkin.

One aspect of the present invention provides a method of preparing the aforementioned liquid-absorbing material, comprising:

bringing the polymer fiber into contact with the absorbent particle and partially melting the polymer fiber by heating, so as to bond the absorbent particle to the polymer fiber after the polymer fiber is cooled and cured.

Optionally, the bringing the polymer fiber into contact with the absorbent particle and partially melting the polymer fiber by heating comprises:

spraying or carding a mixture of the polymer fiber and the absorbent particle, and heating the same to partially melt the polymer fiber.

Optionally, the bringing the polymer fiber into contact with the absorbent particle and partially melting the polymer fiber by heating comprises:

melting and spraying a thermoplastic material to form the polymer fiber, and spraying the absorbent particle toward the polymer fiber still in a partially molten state.

### BRIEF DESCRIPTION OF THE DRAWINGS

FIG. 1 is a structural diagram of an absorbent core according to an embodiment of the present invention;

FIG. 2 is a schematic block diagram of a method of preparing an absorbent core according to an embodiment of the present invention;

FIG. 3 is a schematic block diagram of another method of preparing an absorbent core according to an embodiment of the present invention;

FIG. 4 is a comparison diagram of test results of the absorbability of absorbent cores according to an embodiment of the present invention and in a comparative example;

FIG. 5 is a comparison diagram of test results of the penetrability of absorbent cores according to an embodiment of the present invention and in a comparative example;

FIG. 6 is a comparison diagram of test results of the rewetting of absorbent cores according to an embodiment of the present invention and in a comparative example; and

FIG. 7 is a comparison diagram of test results of the spread in absorbent cores according to an embodiment of the present invention and in a comparative example.

The reference numerals are as follows: 1. polymer fiber; 2. absorbent particle; 31. hydrophilic nonwoven fabric; 32. hydrophobic nonwoven fabric; 51. mixing device; 52. infrared heating device; 61. spinning device; 62. blowing device; 71. unwinding device; and 72. cooling roller.

### DETAILED DESCRIPTION

In order to allow a person of skill in the art to better comprehend technical solutions of the present invention, the present invention is further described in detail below in combination with accompanying drawings and particular embodiments.

### Liquid-Absorbing Material and Absorbent Core

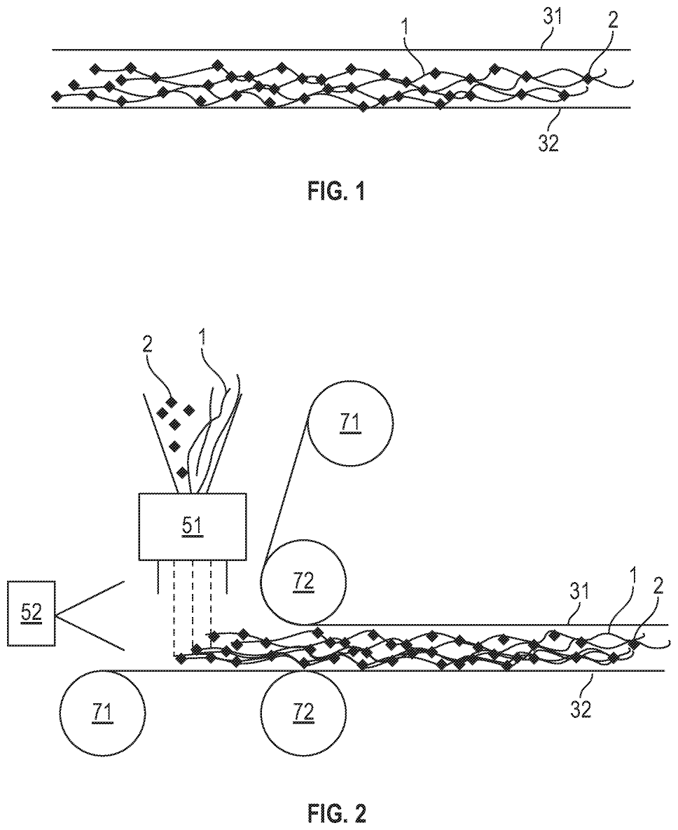

Referring to FIG. 1, an embodiment of the present invention provides a liquid-absorbing material. The liquid-absorbing material is capable of absorbing a liquid such as water.

Specifically, the liquid-absorbing material comprises:

a polymer fiber 1 comprising a polymer material capable of melting when heated, wherein the polymer fiber 1 has been partially molted; and

an absorbent particle 2 having liquid absorbing capacity, wherein when the polymer fiber 1 is in the partially molten state, the absorbent particle 2 is in contact with the polymer fiber 1, thereby bonding to the polymer fiber 1 after the polymer fiber 1 is cured.

Referring to FIG. 1, the liquid-absorbing material according to an embodiment of the present invention comprises the polymer fiber 1. The polymer fiber 1 comprises a polymer material (usually consisting of polymer materials). The polymer material may be brought into a molten state when heated to a certain temperature, thus belonging to thermoplastic materials. Moreover, the polymer fiber 1 in the liquid-absorbing material according to an embodiment of the present invention has been in a partially molten state. That is, surface layers of the polymer fiber 1 have been in a molten state while the fiber morphology is still maintained as a whole. Therefore, the surface layers of the polymer fiber 1 is fused or the like in the molten state, so the polymer fiber 1 has a specific morphology after being cooled and cured.

Optionally, the polymer fiber 1 is a fiber web.

The fiber web refers to fibers having a structure similar to a web formed by the polymer fiber 1 (thermoplastic materials) through a certain process. For example, the polymer fiber 1 comes into contact with each other in a partially molten state, such that surface layers at partial positions are fused together and further form a web structure after being cooled and cured.

Specifically, the polymer fiber 1 comprises at least one of a spunlaid web, a hot air-laid web and a melt blown web. That is, the forming process thereof is specifically carding, spunbonding, melt blowing, and the like.

The liquid-absorbing material further comprises an absorbent particle 2 (for example, a plurality of absorbent particle 2). The absorbent particle 2 refers to a particle consisting of a material capable of absorbing a liquid such as water. The absorbent particle 2 is linked (bonded by melting) to the polymer fiber 1 and thus has a stable linkage to the polymer fiber 1. The term “bonded by melting” means that, when the polymer fiber 1 is partially melted, the absorbent particle 2 is in contact with the polymer fiber 1 and (partially) enters into a molten surface layer of the polymer fiber 1, such that after the polymer fiber 1 is cooled and cured, the absorbent particle 2 and the polymer fiber 1 are fused and bonded together at least at partial positions.

The polymer fiber 1 may have a very small diameter and a very low density. For example, a meltblown polypropylene fiber web may have a diameter of approximately 2 micrometers, which is only 10% of the diameter of a cotton fiber, and a density of only 0.9 to 0.91 g/cm³, which is only about 60% of the density of a cotton fiber. Therefore, the liquid-absorbing material is softer, more air-permeable, lighter, and more comfortable.

Further, since the polymer fiber 1 is a thermoplastic polymer material (plastic material), the polymer fiber 1 is low in cost and can be remelted for recycling, and is thus environmentally friendly.

At the same time, in the liquid-absorbing material, the absorbent particle 2 is stably bonded (bonded by melting) to the polymer fiber 1, so the amount of the absorbent particle 2 effectively carried in the polymer fiber 1 of the same quality is larger. That is, the content of the absorbent particle 2 in the liquid-absorbing material may be higher, whereby the liquid-absorbing material has a better absorption capabilities. At the same time, the morphology of the liquid-absorbing material is more stable, and separation and looseness are less likely to occur.

Optionally, the polymer fiber 1 has a fineness between 1 micrometer and 30 micrometers.

In order to make the liquid-absorbing material softer and more air-permeable, the fineness (diameter) of the polymer fiber 1 is preferably from 1 micrometer to 30 micrometers, and more preferably from 5 micrometers to 20 micrometers.

Optionally, the polymer fiber 1 is a polypropylene elastomeric fiber or a polyester elastomeric fiber. Optionally, the absorbent particle 2 is a super absorbent polymer (SAP) particle, and further, the super absorbent polymer particle consists of super absorbent resin particles (such as sodium polyacrylate particles).

Polypropylene (PP) or polyester materials have suitable density, hardness, melting point, etc., and are low in cost and non-toxic, thus the above polymer fiber 1 can be composed of the elastomeric materials thereof.

The absorbent particle 2 is preferably composed of a super absorbent polymer, more preferably a super absorbent resin, and specifically, sodium polyacrylate materials or the like.

Sodium polyacrylate is also a polypropylene-based material and therefore can be better bonded to polypropylene polymer fiber.

Optionally, the absorbent particle 2 is composed of a high molecule weight polymer material, and the absorbent particle 2 has a melting point higher than that of the polymer fiber 1.

That is, the absorbent particle 2 can also be composed of a high molecule weight polymer material (particularly a thermoplastic high polymer material) and has a melting point higher than that of the material (inevitably a thermoplastic material) of the polymer fiber 1. Thus, when the absorbent particle 2 are bonded to the polymer fiber 1, the polymer fiber 1 must be partially melted while maintaining the overall morphology thereof, and the relatively small absorbent particle 2 is not melted to avoid damaging the morphology thereof, ultimately ensuring good bonding of the polymer fiber 1 and the absorbent particle 2.

Optionally, the absorbent particle 2 has a particle size between 200 micrometers and 600 micrometers.

In order to balance the amount of addition, absorption capability, and the like, the absorbent particle 2 may have a particle size between 200 micrometers and 600 micrometers, preferably between 350 micrometers and 450 micrometers.

Optionally, the liquid-absorbing material comprises 1 wt % to 95 wt % of the absorbent particle 2.

As described above, the content range of the absorbent particle 2 in the above liquid-absorbing material may be larger. The absorbent particle 2 in the liquid-absorbing material may be between 1 wt % and 95 wt %, preferably between 70 wt % and 90 wt %. By contrast, in existing liquid-absorbing materials using wood pulp fibers, the mass percentage of the absorbent particle 2 can generally only be 20% to 35%.

An embodiment of the present invention further provides an absorbent core, comprising:

an absorbent layer consisting of any of the aforementioned liquid-absorbing materials.

That is, the above liquid-absorbing material may be made “layered” so as to be used as an absorbent layer of an absorbent core in absorbent products such as disposable diapers or sanitary napkins.

Certainly, it should be understood that in the absorbent layer above, the lengthwise direction of the polymer fiber 1 should be mainly distributed in the face in which the absorbent layer is located, and may also be distributed mainly along a specific direction in the face.

Optionally, the absorbent layer has a thickness between 0.2 mm and 30 mm, preferably between 0.2 mm and 20 mm. Optionally, the absorbent layer has a grammage between 10 gsm and 1000 gsm (gram per square meter), preferably between 50 gsm and 800 gsm, more preferably between 200 gsm and 800 gsm.

Since the liquid-absorbing material used in the absorbent core has a better water absorption capability, the absorbent core may have a smaller thickness and a lower grammage compared with an existing absorbent core, such that the product is lighter, thinner, more air-permeable, and more comfortable.

In order to adjust the air permeability of the absorbent core, the fineness of the polymer fiber 1 can be adjusted, or the absorbent layer can be perforated.

Optionally, the absorbent core further comprises: a hydrophilic nonwoven fabric 31 and a hydrophobic nonwoven fabric 32 respectively arranged on both sides of the absorbent layer.

Referring to FIG. 1, in the above absorbent core, the hydrophilic nonwoven fabric 31 and the hydrophobic nonwoven fabric 32 can be respectively arranged on both sides of the absorbent layer to relatively fix the absorbent layer. When the absorbent core is used in an absorbent product such as a disposable diaper or a sanitary napkin, the hydrophilic nonwoven fabric 31 is usually oriented toward the side of a liquid source (i.e., a side close to a human body) so that the liquid can smoothly enter the absorbent layer. The hydrophobic nonwoven fabric 32 is located on the side away from the human body, so as to prevent the absorbed liquid from leaking from the side.

### Method of Preparing Liquid-Absorbing Material and Absorbent Core

Referring to FIGS. 1 to 3, an embodiment of the present invention provides a method of preparing any of the aforementioned liquid-absorbing materials, comprising:

bringing the polymer fiber 1 into contact with the absorbent particle 2 and partially melting the polymer fiber 1 by heating, so as to bond the absorbent particle 2 to the polymer fiber 1 after the polymer fiber 1 is cooled and cured.

That is, the polymer fiber 1 can be brought into contact with the absorbent particle 2 when being partially melted, so that, after cooling and curing, the absorbent particle 2 is bonded (bonded by melting) to the polymer fiber 1, forming the aforementioned liquid-absorbing material.

In the preparation method above, it is only necessary to ensure that the polymer fiber 1 is brought into contact with the absorbent particle 2 while in a partially molten state, and the specific manner of achieving such condition varies. For example, the polymer fiber 1 and the absorbent particle 2 may be mixed and then heated to partially melt the polymer fiber 1. In another example, the polymer fiber 1 may be partially melted first, and then the absorbent particle 2 may be mixed therewith. The specific preparation method may be as follows:

### 1) Preparation Method 1

Specifically, as a method according to an embodiment of the present invention, the preparation method of the above liquid-absorbing material may comprise:

spraying or carding a mixture of the polymer fiber 1 and the absorbent particle 2, and heating the same to partially melt the polymer fiber 1.

That is, referring to FIG. 2, the already fibrous polymer fiber 1 and the absorbent particle 2 may be put into a mixing device 51 and mixed, and sprayed from the mixing device 51 or carded. The mixture in the process of spraying or carding is heated through an infrared heating device 52, so that the polymer fiber 1 is partially melted. Thus, after curing, the polymer fiber 1 and the absorbent particle 2 are bonded together (bonded by melting).

Of course, if the absorbent core is to be prepared, subsequently, the hydrophilic nonwoven fabric 31 and the hydrophobic nonwoven fabric 32 may be respectively unwound by two unwinding devices 71 to surfaces of both sides of the liquid-absorbing material, and the product is cooled by two cooling rollers 72.

### 2) Preparation Method 2

Specifically, as another method according to an embodiment of the present invention, the preparation method of the above liquid-absorbing material may comprise: melting and spraying a thermoplastic material to form the polymer fiber 1, and spraying the absorbent particle 2 toward the polymer fiber 1 still in a partially molten state.

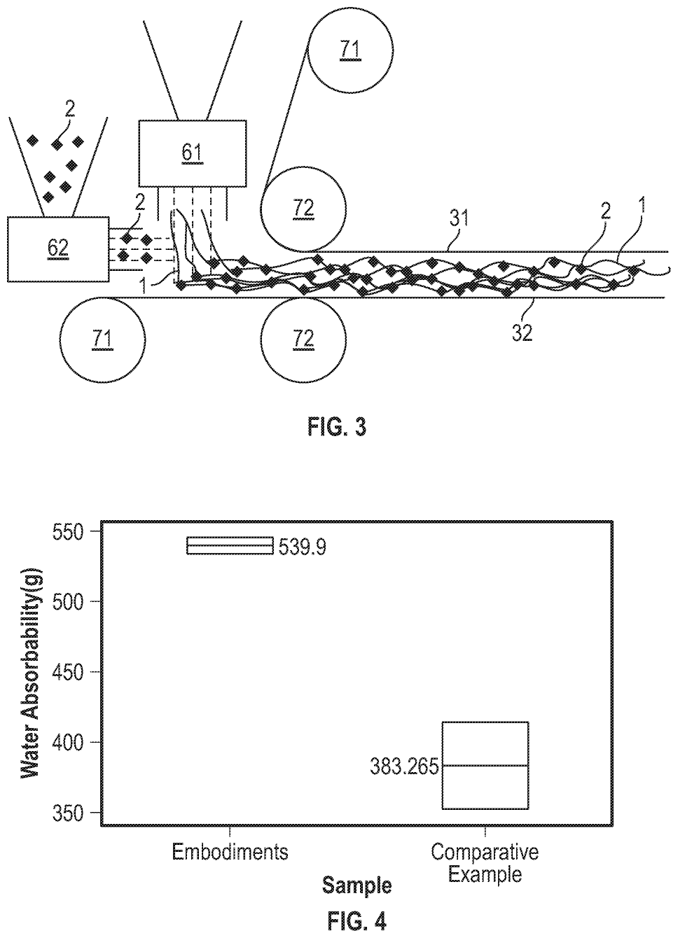

That is, referring to FIG. 3, a raw material of the thermoplastic material may be added to a spinning device 61 to spray a flow of liquid in a molten state from the spinning device 61, and the flow of liquid is cooled and cured into the polymer fiber 1. At the same time, the absorbent particle 2 is introduced into a blowing device 62 and blown toward the polymer fiber 1 sprayed from the spinning device 61 and not completely cured (i.e., partially melted), thereby bonding (bonding by melting) the polymer fiber 1 to the absorbent particle 2 after the polymer fiber is completely cured.

Certainly, if the absorbent core is to be prepared, subsequently, the hydrophilic nonwoven fabric 31 and the hydrophobic nonwoven fabric 32 may be respectively unwound by two unwinding devices 71 to surfaces of both sides of the liquid-absorbing material, and the product is cooled by two cooling rollers 72.

### Absorbent Product

An embodiment of the present invention provides an absorbent product, comprising: any of the aforementioned absorbent cores.

That is, the above absorbent core may be used in a specific absorbent product. For example, the absorbent product may be any one of a disposable nappy, a disposable diaper, a pair of disposable training pants, a sanitary napkin and a napkin.

Certainly, depending on the specific absorbent product, the absorbent core therein may have different forms. For example, for napkins, the absorbent core may not comprise a nonwoven fabric.

Certainly, the absorbent product may also be in other forms, and may comprise other known structures such as a permeable layer, a bonding layer and an anti-side leakage layer, which will not be described in detail herein.

### Specific Embodiments

Products according to embodiments of the present invention and in comparative examples were tested and the performances thereof were compared below.

### 1) Raw Materials

Raw materials used in the products according to embodiments of the present invention and in comparative examples are described below:

Absorbent Particle: super absorbent resin particle from Sanyo; model: Sanfresh YH-2; melting point: 200° C.; decomposition temperature: greater than 300° C.; particle size: 100 mesh;

Raw Materials of Polymer Fiber (Fiber Webs): hydrophilic polyester elastomer from DuPont; model: G3548L; melting point: 170° C.; decomposition temperature: greater than 300° C.; fineness: 400 deniers;

Hydrophilic Nonwoven Fabric: from Beautiful NW Company; model: SSS, 20 grams.

Comparative Example 1: an absorbent core (excluding a nonwoven fabric) was taken out of a disposable diaper product of a commercially available brand as the Comparative Example 1. Specifically, materials of the absorbent core comprise fluff pulp (wood pulp), and super absorbent resin particles of a commercially available brand.

Comparative Example 2: an absorbent core (excluding a nonwoven fabric) was taken out of a disposable diaper product of another commercially available brand as the Comparative Example 2. Specifically, materials of the absorbent core comprise fluff pulp (wood pulp), and super absorbent resin particles of a commercially available brand.

### 2) Performance Test Method

For the products according to embodiments of the present invention and in comparative examples, the performances thereof were tested in accordance with the following methods:

### (1) Absorption Capability (Water Absorption Capability)

Tests were conducted in accordance with the following methods:

A. A sample of a predetermined weight was cut out;

B. Normal saline was poured into a container;

C. The sample was placed in the container with the entire surface saturated and soaked for 5 minutes;

D. The sample was removed from the container, clamped with a folding clip, and suspended vertically for 1 minute;

E. The clip was loosened, the sample was put on a scale, and the weight thereof was recoded as the weight after water absorption; and

F. The test result of absorption capability (in grams) was obtained by subtracting the weight before water absorption from the weight after water absorption (substantially the weight gain after water absorption of the sample), wherein each of the embodiments or comparative examples was separately tested with 3 different samples.

### (2) Penetrability, Rewetting and Spread

The samples were tested for penetrability, rewetting, and spread by the following methods:

A. A jig was placed on the top surface of a sample. The jig was a transparent plate having a thickness of 25.4 mm and a well of 25.4 mm in diameter. The well thereof was connected to the upper surface of the sample;

B. 10 grams of normal saline was weighed (stained with bright blue pigment);

C. The 10 grams of normal saline was poured into the well of the jig rapidly;

D. The time (in seconds) taken from the complete pouring of the normal saline to the full penetration thereof into the sample was recorded as a parameter indicating the penetrability;

E. After holding for 5 minutes, the spreading range of the normal saline in the sample was observed for evaluation of the spread;

F. 10 sheets of filter paper were placed between the upper surface of the sample and the bottom of a fixture, wherein the 10 sheets of filter paper have been pre-weighed, and the result of the weighing was an initial weight;

G. A weight of 2 kg was placed on the top of the fixture, and the weight, the fixture and the 10 sheets of filter paper were removed after 15 seconds; and

H. The 10 sheets of filter paper were weighed and the weight was taken as a final weight.

The weight difference (in grams) obtained by subtracting the initial weight from the final weight was used as a parameter indicating the rewetting.

Each of the embodiments or comparative examples was separately tested using three different samples, each sample was tested three times in succession, and the samples were set aside without a fixture for 2 minutes before the second and third tests.

### (3) Thickness

The thicknesses of the samples before and after water absorption were separately measured using a micrometer caliper.

### (4) Appearance

The appearances of the samples before and after water absorption were separately observed.

### Embodiment 1

Using the above Preparation Method 1, an absorbent core was prepared in accordance with the parameters in Table 1 below as the Embodiment 1.

### Embodiment 2

Using the above Preparation Method 2, an absorbent core was prepared in accordance with the parameters in Table 1 below as the Embodiment 2.

TABLE 1
Comparison of Parameters and Performances of Absorbent Cores

| | | Comparative | | Comparative |
|---|---|---|---|---|
| | Embodiment 1 | Example 1 | Embodiment 2 | Example 2 |
| Mass content of absorbent | 94% | 30% | 79.5% | 45% |
| particle in the absorbent core | | | | |
| Mass content of polymer | 6% | 70% | 20.5% | 55% |
| fiber in the absorbent core | | | | |
| Grammage (gsm) of the | 320 | 700 | 340 | 800 |
| absorbent core | | | | |
| Thickness (mm) of the | 2 | 5 | 2 | 5 |
| absorbent core before water | | | | |
| absorption | | | | |
| Thickness (mm) of the | 5 | 25 | 5 | 25 |
| absorbent core after water | | | | |
| absorption | | | | |
| Appearance of the absorbent | The absorbent particle was | The absorbent particle was | The absorbent particle was | The absorbent particle was |
| core after water absorption | bonded to the polymer fiber | separated from the polymer | bonded to the polymer fiber | separated from the polymer |
| | and the absorbent core | fiber and the absorbent core | and the absorbent core | fiber and the absorbent core |
| | was lamellar | was loosely packed | was lamellar | was loosely packed |

It can be seen from the table above that, in the absorbent core according to the embodiment of the present invention, the content of absorbent particle is much larger compared with the existing absorbent core, so that a better absorption performance can be obtained.

When the total weights are the same, the thickness of the absorbent core according to the embodiment of the present invention is much smaller compared with the existing absorbent core, indicating that the absorbent core is lighter and thinner, and has better air permeability than the existing absorbent core.

At the same time, after full water absorption, the absorbent core according to the embodiment of the present invention remains substantially intact in morphology, while the existing absorbent core is obviously separated and loose, indicating that the absorbent core according to the embodiment of the present invention has a better shape preserving property.

The samples of the Embodiment 1 and Comparative Example 1 above were subjected to an absorbability (water absorbability) test, and the results thereof are shown in FIG. 4. Each square represents the test results of a product in one embodiment or comparative example, the upper and lower edges of the square represent the maximum and minimum values of the test results of three samples of the product respectively, and the line in the middle of the square represents the mean value of the test results of the three samples of the product.

It can be seen that, in the case of the same grammage, the amount of water absorbed by the absorbent core according to the embodiment of the present invention is much larger compared with the existing absorbent core, and the amount of absorbed water has a narrower distribution range, indicating that the absorbent core according to the embodiment of the present invention has better and more stable absorption capabilities.

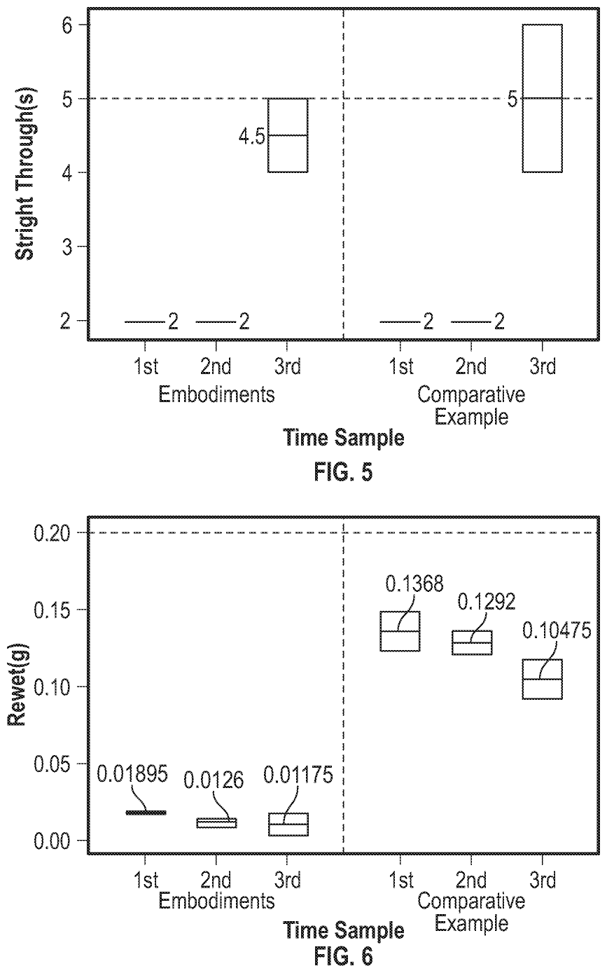

The samples of the Embodiment 2 and Comparative Example 2 above were subjected to a penetrability and a rewetting test, and the results thereof are shown in FIG. 5 and FIG. 6. Each square represents the results of one test of a product in one embodiment or comparative example (each sample was tested for three times, so each product corresponds to three squares), the upper and lower edges of the square represent the maximum and minimum values of the results of one test of the three samples of the product respectively, and the line in the middle of the square represents the mean value of the results of one test of the three samples of the product.

It can be seen that, referring to FIG. 5, in the third test, the penetration time of the absorbent core according to the embodiment of the present invention is much less compared with the existing absorbent core, and the penetration time has a narrower distribution range, indicating that the absorbent core according to the embodiment of the present invention has a better and more stable penetrability.

It can be seen that, referring to FIG. 6, in each test, the rewetting amount of the absorbent core according to the embodiment of the present invention is much less compared with the existing absorbent core, and the rewetting amount has a narrower distribution range, indicating that the absorbent core according to the embodiment of the present invention has a better rewetting, and the rewetting is more stable.

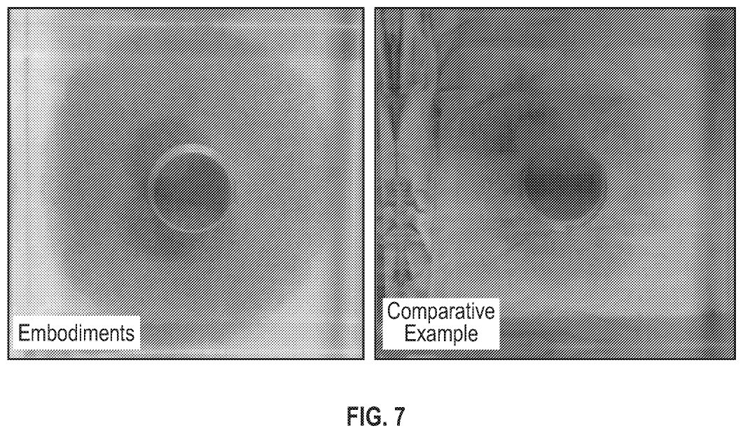

The samples of the Embodiment 2 and Comparative Example 2 above were subjected to a spread test, and the results thereof are shown in FIG. 7.

It can be seen that, compared with the existing absorbent core, the spreading range of water in the absorbent core according to the embodiment of the present invention is obviously larger and has a uniform distribution, indicating that the absorbent core according to the embodiment of the present invention makes water spread in a more uniform and greater range and thus has a better spread.

It can be understood that, the above embodiments are only exemplary embodiments employed for illustration of principles of the present invention, and do not limit the present invention. For those of ordinary skill in the art, various variations and modifications may be made without departing from the spirit and essence of the present invention, which variations and modifications are also considered as falling within the protection scope of the present invention.

1. A liquid-absorbing material, comprising:
a polymer fiber, comprising a polymer material capable of melting when heated, wherein the polymer fiber has been partially molted; and
an absorbent particle having liquid absorbing capability, wherein when the polymer fiber is in a partially molten state, the absorbent particle is in contact with the polymer fiber, thereby bonding to the polymer fiber after the polymer fiber is cured.

2. The liquid-absorbing material according to claim 1, wherein
the polymer fiber comprises a fiber web, the fiber web comprising at least one of a spunlaid web, a hot air-laid web and a melt blown fiber web.

3. The liquid-absorbing material according to claim 1, wherein
the polymer fiber has a fineness between 1 micrometer and 30 micrometers.

4. The liquid-absorbing material according to claim 1, wherein
the polymer fiber comprises polypropylene elastomeric fibers or polyester elastomeric fibers.

5. The liquid-absorbing material according to claim 1, wherein
the absorbent particle comprises a super absorbent polymer particle.

6. The liquid-absorbing material according to claim 5, wherein
the super absorbent polymer particle comprises sodium polyacrylate particles.

7. The liquid-absorbing material according to claim 1, wherein
the absorbent particle has a particle size between 200 micrometers and 600 micrometers.

8. The liquid-absorbing material according to claim 1, wherein
the liquid-absorbing material comprises 1 wt % to 95 wt % of the absorbent particle.

9. The liquid-absorbing material according to claim 1, wherein
the liquid-absorbing material comprises 70 wt % to 90 wt % of the absorbent particle.

10. The liquid-absorbing material according to claim 1, wherein
the absorbent particle is composed of a high molecular weight polymer material, and the absorbent particle has a melting point higher than that of the polymer fiber.

11. An absorbent core, comprising:
an absorbent layer composed of the liquid-absorbing material according to claim 1.

12. The absorbent core according to claim 11, wherein
the absorbent layer has a thickness between 0.2 mm and 30 mm.

13. The absorbent core according to claim 11, wherein
the absorbent layer has a grammage between 10 gsm and 1000 gsm.

14. The absorbent core according to claim 11, further comprising:
a hydrophilic nonwoven fabric and a hydrophobic nonwoven fabric respectively arranged on two sides of the absorbent layer.

15. An absorbent product, comprising:
the absorbent core according to claim 11.

16. The absorbent product according to claim 15, wherein
the absorbent product is any one of a disposable nappy, a disposable diaper, a pair of disposable training pants, a sanitary napkin, and a napkin.

17. A method of preparing a liquid-absorbing material, wherein the liquid-absorbing material is the liquid-absorbing material according to claim 1, and the method of preparing the liquid-absorbing material comprises:
bringing the polymer fiber into contact with the absorbent particle and partially melting the polymer fiber by heating, so as to bond the absorbent particle to the polymer fiber after the polymer fiber is cooled and cured.

18. The method of preparing the liquid-absorbing material according to claim 17, wherein the bringing the polymer fiber into contact with the absorbent particle and partially melting the polymer fiber by heating comprises:
spraying or carding a mixture of the polymer fiber and the absorbent particle, and heating the same to partially melt the polymer fiber.

19. The method of preparing the liquid-absorbing material according to claim 17, wherein the bringing the polymer fiber into contact with the absorbent particle and partially melting the polymer fiber by heating comprises:
melting and spraying a thermoplastic material to form the polymer fiber, and spraying the absorbent particle toward the polymer fiber still in a partially molten state.
